(19) United States
(12) Patent Application Publication
(10) Pub. No.: US 2006/0058665 A1
(43) Pub. Date: Mar. 16, 2006

(54) Noninvasive method of ultrasound wound evaluation

(75) Inventor: James Vincent Chapman, Carmel, IN (US)

Correspondence Address: Chad A Coberly, 9318 Ricker St., Richland, MI, 49083, US

(73) Assignee: Biosound, Inc.

(21) Appl. No.: 11/207,246

(22) Filed: Aug. 19, 2005

Related U.S. Application Data
(60) Provisional application No. 60/602,732, filed on Aug. 19, 2004.

(51) Int. Cl. A61B 8/12

(52) U.S. Cl. 600/439

(57) ABSTRACT

A noninvasive method of ultrasound wound evaluation whereby a health care professional fills a wound with a sterile coupling gel, covers the wound with a non-adhesive couplant sheet, scans the wound with a ultrasound transducer that has been covered with a sterile sheath, thereby producing measurement data about the wound including the diameter, the perimeter and the depth, converting the data into a report package to be utilized by a health care professional to characterize the wound.

### PRIORITY

The following non-provisional patent application claims priority to provisional application U.S. Ser. No. 60/602,732.

### FIELD OF THE INVENTION

The present invention relates generally to the field of wound evaluation using ultrasound, and more specifically, to a method of measuring and evaluating a wound present on a human body by methodically following a procedure to protect and prepare the wound for analysis by a healthcare professional utilizing ultrasonic waves.

### BACKGROUND OF INVENTION

Diagnostic ultrasound has proven to be an invaluable aid in the appreciation of the extent and damage caused by ulcerations. With deep, tunneling ulcerations, the method of clinically evaluating a wound was to insert a probe into the wound and “sound” the surfaces to get a general idea of the ulcers extent. This resulted in an imprecise measurement. With diagnostic ultrasound, the clinician can visualize the wound's extent on different body planes, measure the base of the ulceration to bone and other important structures, and appreciate what structures are violated and compromised.

In order to document the progress or regression in the size of the wound, the clinician can accurately measure, length, width, depth, and circumference of the wound, and compare to previous scans. Diagnostic ultrasound of wounds has taken the evaluation of ulcerations to a new level.

Previously, methods have been utilized to analyze wounds with ultrasound by applying a coupling medium to the wound with a protective covering held in place by adhesives or other intrusive attachment means. While these methods have proven to be effective, the adhesive protective layer can damage the surrounding skin to the wound or further damage the wound itself. Other methods of applying ultrasonic waves to analyze wounds by not covering the wound can result in contamination of the wound site or ineffective application of the ultrasound waves due to not having an effective coupling medium in which to receive the ultrasonic waves. There exists a need for a method that can utilize existing products on the market to achieve the protection for the wound while promoting the application of the ultrasound technology.

One previous ultrasound wound evaluation protocol, U.S. Pat. No. 6,193,658, Wendelken, employed a sterile, adhesive barrier with 10 cm×11.4 cm dimensions. Measurements are taken on the long and short axis as well as the depth and distance from the base of the ulceration to the bone. Once these calculations are made, the length, width and depth are multiplied to gain a volumetric measurement of the lesion.

The drawback to this system is the adhesive barrier, which is contraindicated in weak, friable skin adjacent to ulceration. In addition, the barrier is often too small to adequately protect the entirety of the lower extremity wounds. Also the method of multiplying the length, width and depth of a wound to obtain a volumetric measurement is often inaccurate. Wounds are usually irregular, not perfectly square or rectangular in geometry. Wounds are always irregular, and many tunnel, extending the length and width, giving measurements that will greatly exaggerate the error of a simple length, width, depth calculation. In addition, there is no protocol for the exact location (within the body planes) these measurements are to be taken.

### ADVANTAGES OF INVENTION

The present invention overcomes the prior art by better protecting the wound by not utilizing an adhesive layer on the ultrasound media film. The present invention also allows for better coverage of the wound area by not restricting the size of the film layer by the adhesive edges. The invention uses more accurate measurement techniques to estimate the volume of a wound than previous methods.

### SUMMARY OF INVENTION

The invention comprises a method for evaluating the extent of wounds, using a non-adhesive based couplant layer and ultrasound for image acquisition and applying a computer based measurement package of an ultrasound equipment system to measure either the wounds linear parameters, or the circumference and depth for volume determination. This information is used for the medical specialist to evaluate the efficacy of a particular treatment in the reduction of the wound area.

### BRIEF DESCRIPTION OF THE DRAWINGS

FIG. 1 is a perspective, schematic view of a computer controlled ultrasound image system in accordance with and embodying the present invention.

FIG. 2 is a cross sectional view of a typical wound present on the surface of a human body with the wound filled with a couplant gel and covered by a non-adhesive couplant sheet.

FIG. 3. is a view of a wound covered by a non-adhesive couplant sheet of sufficient size to cover the entire wound.

FIG. 4. is a perspective view of a computer controlled ultrasound system.

### DESCRIPTION OF PRACTICAL EMBODIMENTS

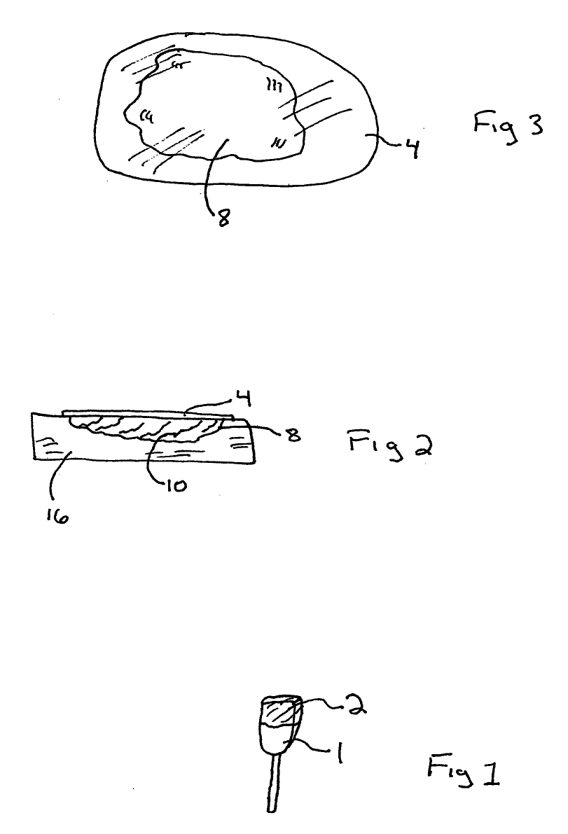

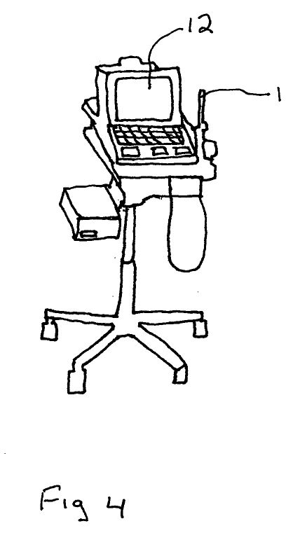

With reference to the drawings, and specifically FIGS. 1-4, The method uses a commercially available ultrasound couplant sheet 4, of sufficient size to completely cover a wound 8 in a human body after it is filled with a sterile coupling gel 10. A linear or convex array ultrasound transducer 1 is scanned over the covered wound 8 to image the extent of the wound 8. The cover 4 is not sealed with adhesive edges area, as there is a potential to cause damage to the sensitive skin and tissue layers 16 area adjacent to the wound. The cover 4 is held in place over the wound 4 by making contact with the coupling gel 10. To prevent contamination of the wound during the scan the ultrasound probe 1, which may be a broad bandwidth linear or convex array transducer, is covered with a commercially available sterile sheath 2. The ultrasound transducer 1, is connected to a computer controlled ultrasound system 12 which includes a keyboard, joystick, and other clinicain operated controls. The computer controlled image system 12 processes the data signal received from the ultrasound transducer 1 and displays an image and measurement data regarding the wound 8 such that the clinician can utilize the information to assist in conducting further clinical analysis. The computer controlled ultrasound system 12 allows the clinician to perform area length or circumference measurements to evaluate the dimensions of the wound 8. The depth of the wound may also be measured, however, the measurements do not include a volume calculation based on the measurements due to several geometric assumptions that can not be verified in the irregularities of any given wound. In another emodiment of the invention, a perimeter of the wound is measured using the ultrasound system 12.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1. A noninvasive method of ultrasound wound evaluation comprising:
filling a wound with a sterile coupling gel;
covering said wound with a couplant sheet;
scanning said couplant sheet with an ultrasound transducer to measure the diameter of said wound;
transmitting the measurements obtained to a computer controlled processing unit;
configuring the measurement data into a report package to be utilized by a physician to assist in the diagnosis of the wound;
wherein said couplant sheet is non-adhesive thereby protecting the wound during measurement without causing further trauma to the wound.

2. The method of claim 1 further comprising the step of:
measuring the perimeter of said wound wherein said wound is irregular in shape;
adding said perimeter measurement data to said report package.

3. The method of claim 1 further comprising the step of:
measuring the depth of said wound;
adding said depth measurement data to said report package.

4. The method of claim 1 further comprising the step of:
covering said ultrasound transducer with a sterile sheath in order to maintain a sterile field around the wound during the scanning process.

5. The method of claim 1 wherein said ultrasound transducer is a broad bandwidth linear transducer.

6. The method of claim 1 wherein said ultrasound transducer is a convex array transducer.

7. A noninvasive method of ultrasound wound evaluation comprising:
providing a kit to measure wounds, said kit comprising non-adhesive ultrasound couplant sheets, sterile coupling gel, and protective transducer sheaths;
applying said sterile coupling gel to fill a wound;
covering said wound with a couplant sheet;
scanning said couplant sheet with an ultrasound transducer to measure the diameter of said wound;
transmitting the measurements obtained to a computer controlled processing unit;
configuring the measurement data into a report package to be utilized by a physician to assist in the diagnosis of the wound;
wherein said couplant sheet is non-adhesive thereby protecting the wound during measurement without causing further trauma to the wound.

8. The method of claim 7 further comprising the step of:
measuring the perimeter of said wound wherein said wound is irregular in shape;
adding said perimeter measurement data to said report package.

9. The method of claim 7 further comprising the step of:
measuring the depth of said wound;
adding said depth measurement data to said report package.

10. The method of claim 7 further comprising the step of:
covering said ultrasound transducer with a sterile sheath in order to maintain a sterile field around the wound during the scanning process.

11. The method of claim 7 wherein said ultrasound transducer is a broad bandwidth linear transducer.

12. The method of claim 7 wherein said ultrasound transducer is a convex array transducer.
